(19) United States
(12) Patent Application Publication
(10) Pub. No.: US 2005/0077390 A1
(43) Pub. Date: Apr. 14, 2005

(54) Dispensing cap with pressure lever

(76) Inventors: Lin Ching Chi, Guangdong (CN); Sun Quan, Guangdong (CN)

Correspondence Address: FAY, SHARPE, FAGAN, MINNICH & MCKEE, LLP, 1100 SUPERIOR AVENUE, SEVENTH FLOOR, CLEVELAND, OH, 44114, US

(21) Appl. No.: 10/499,190

(22) PCT Filed: Mar. 17, 2003
(86) PCT No.: PCT/CN03/00193

(30) Foreign Application Priority Data
Dec. 27, 2002 (CN) 02250698.5

(51) Int. Cl. B05B 9/04; A61M 11/02; B05B 1/26

(52) U.S. Cl. 239/373; 239/533.1; 239/461

(57) ABSTRACT

The present invention relates to a container's spray head, and in particular, to a convenient and laborsaving pressure activated spray head, which comprises a main body, an air pressure tube, a pressure rod, a spray tube and a connecting sleeve, the air pressure tube is inserted into and disposed in the main body; the pressure rod that is hinged to the main body presses against the upper surface of a pressure head of the air pressure tube; a baffle disposed on the air pressure tube abuts against the upper surface of the connecting sleeve, and the connecting sleeve is embedded in the main body; the spray tube engages with the pressure head of the air pressure tube and communicates with an inner chamber of the air pressure tube. The present invention adopts lever principle, and it lengthens the arm of the applied force. Under the same reactive torque, the longer the arm is, the smaller the applied force is. The present invention decreases the force applied by utilizing the pressure rod and thus could be used more conveniently and more laborsavingly.

### FIELD OF THE INVENTION

The present invention relates to a container spray head, and in particular, to a pressure activated spray head which can be used conveniently and laborsaving.

### BACKGROUND ART

Many commonly used household goods are provided with a spray head of air pressure type, which main function is to draw out the liquid contained in a container through an air pressure tube by utilizing an air pressure principle. For example, most of the spray heads are disposed on the containers such as bath lotion, hand cleaner and the like employing this drawout method of air pressure tube. When use, a pressure head on the air pressure tube is pressed down by hand, forcing air generated during compression process to enter the container. Then, air pressure in the container will increase. Under the pressure effect of this increased air pressure, liquid in the container may be drawn out along the inner chamber of the air pressure tube. The structure of this type may not only be used conveniently, but also decrease a waste of liquid effectively; therefore it has been used widely. But this structure also has some disadvantages. For example, the operational mode of this structure is to press down the pressure head of the air pressure tube directly. And the pressure head is supported on the air pressure tube via a spring whose elastic force is relatively larger generally. Therefore, when a user wants to press down the pressure head, he has to overcome some trouble. When the user is an old man or a child, the situation may be more apparent.

Accordingly, it is the object of the present invention to provide an pressure activated spray head which can be used conveniently and laborsavingly.

### SUMMARY OF THE INVENTION

In order to achieve the aims described above, the present invention adopts such a technical solution that: a pressure activated spray head comprises a main body, an air pressure tube, a pressure rod, a spray tube and a connecting sleeve, the air pressure tube is inserted into and disposed in the main body; the pressure rod that is hinged to the main body pressing against the upper surface of a pressure head of the air pressure tube; a baffle disposed on the air pressure tube abuts against the upper surface of the connecting sleeve, and the connecting sleeve is embedded in the main body; the spray tube engages with the pressure head of the air pressure tube and communicates with an inner chamber of the air pressure tube.

The upper end of the main body has a spherical shape, and two sliding chutes are provided in the upper end of the main body, the pressure rod disposed in one of the sliding chutes is hinged to the upper end via a pivot pin, the spray tube is provided to extend through another sliding chute and threadly engage with the pressure head of the air pressure tube.

The present invention adopts lever principle, and it lengthens the arm of the applied force. Under the same reactive torque, the longer the arm is, the smaller the applied force is. The present invention decreases the force applied by utilizing the pressure rod and thus could be used more conveniently and more laborsavingly.

### BRIEF DESCRIPTION OF THE DRAWINGS

FIG. 1 is a perspective view of a spray head according to the embodiment of the present invention;

FIG. 2 is an exploded diagram showing the spray head in FIG. 1;

FIG. 3 is a sectional diagram showing the spray head in FIG. 1;

FIG. 4 is a sectional diagram showing the operation status of the spray head in FIG. 1.

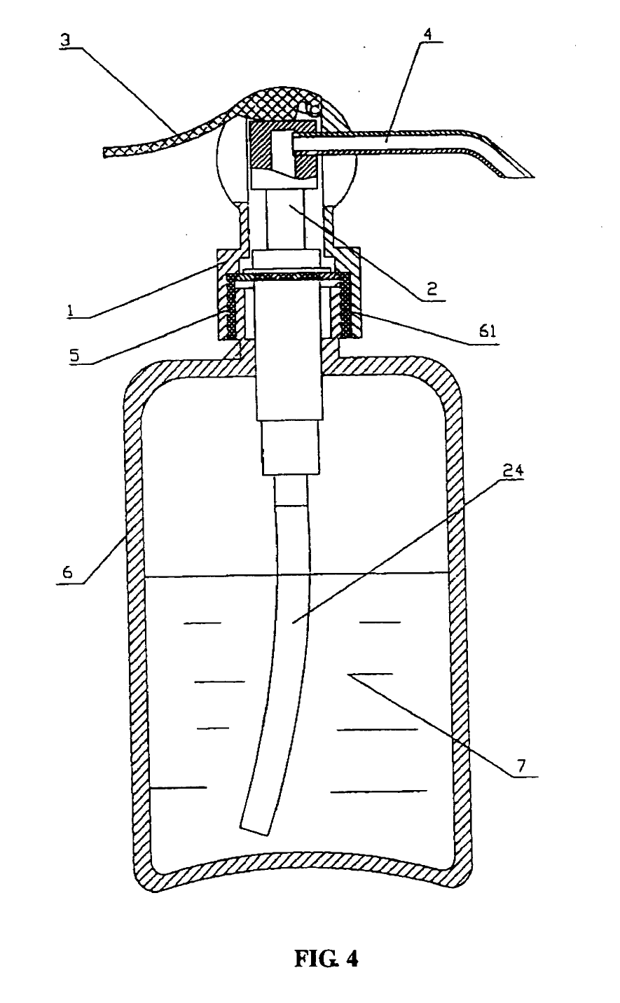

### DETAILED DESCRIPTION OF PREFERRED EMBODIMENTS

The present invention will be obvious by description combined with the following drawings and the preferred embodiments.

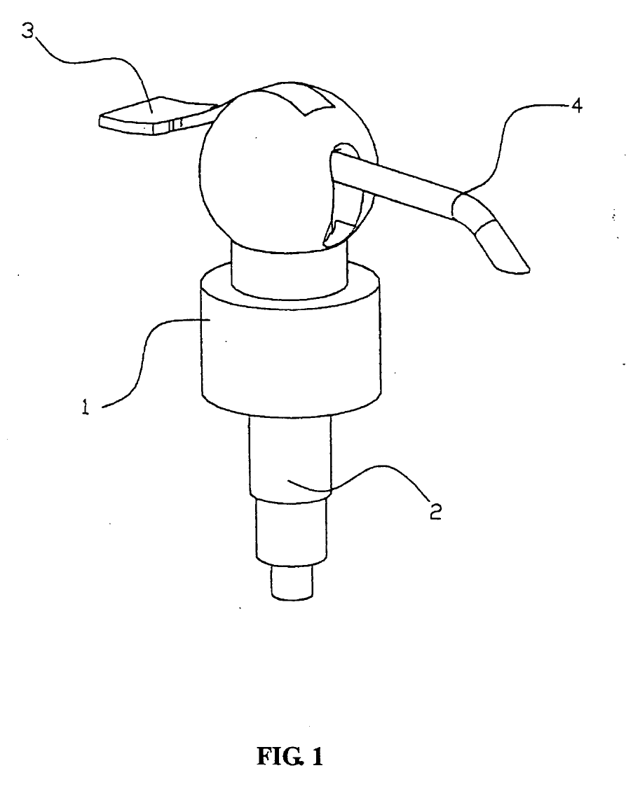

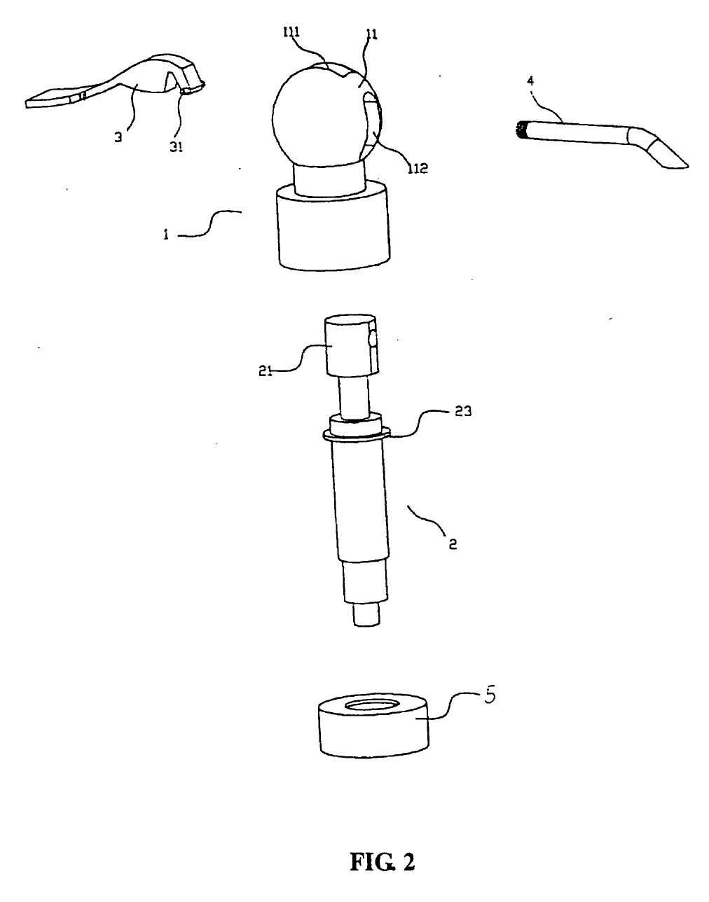

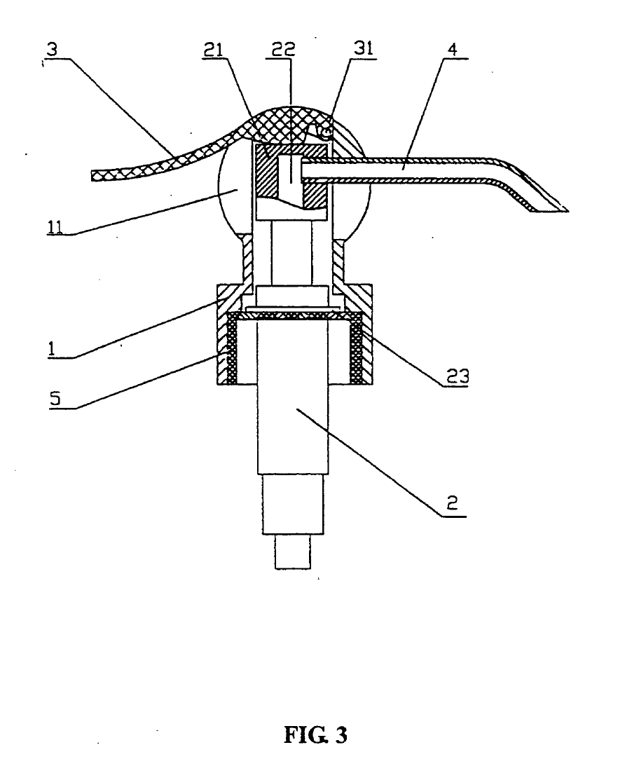

Referring to FIGS. 1, 2 and 3, the spray head comprises: a main body 1, an air pressure tube 2, a pressure rod 3, a spray tube 4 and a connecting sleeve 5.

The interior part of the main body 1 is a cavity. The upper end 11 of the main body 1 has a spherical shape. Two sliding chutes 111 and 112 are provided in the upper end 11 of the main body 1. The pressure rod 3 disposed in the sliding chute 111 is hinged to the upper end 11 via a pivot pin 31. The air pressure tube 2 is inserted into and disposed in the cavity of the main body 1. The pressure rod 3 that is hinged to the main body 1 presses against the upper surface of a pressure head 21 of the air pressure tube 2. A baffle 23 disposed on the air pressure tube 2 abuts against the upper surface of the connecting sleeve 5. To make the connecting sleeve 5 not rotate relative to the main body 1, the connecting sleeve 5 is embedded in the main body 1. The spray tube 4 extends through the sliding chute 112 and threadly engages with the pressure head 21 of the air pressure tube 2. In the meanwhile, the spray tube 4 communicates with an inner chamber 22 of the air pressure tube 2.

A screw thread provided on the inner wall of the connecting sleeve 5 is used to connect with a container 6.

During use, the spray head is fixed on the opening 61 of the container 6 by the screw thread arranged on the connecting sleeve 5. Depending on the size of the container 6 and the amount of the liquid, the lower end of the air pressure tube 2 may be connected with another tube 24, of which the inlet is close to the bottom surface of the container 6. During operation, the pressure rod 3 is pressed down by hand, rotating around the pivot pin 31 and thus pressing the pressure head 21 of the air pressure tube 2 down. Then, air generated during compression process of the air pressure tube 2 will be compressed into the container 6, causing air pressure in the container 6 to increase. Under this increased air pressure, liquid 7 in the container 6 may be drawn out along the inner chamber 24 of the air pressure tube 2 and be sprayed out through the spray tube 4.

1. A pressure activated spray head comprising:
a main body (1), an air pressure tube (2), a pressure rod (3), a spray tube (4) and a connecting sleeve (5), wherein said air pressure tube (2) is inserted into and disposed in said main body (1);
said pressure rod (3) that is hinged to said main body (1) presses against the upper surface of a pressure head (21) of said air pressure tube (2);
a baffle (23) disposed on said air pressure tube (2) abuts against the upper surface of said connecting sleeve (5), and said connecting sleeve (5) is embedded in said main body (1);
said spray tube (4) is formed to engage with said pressure head (21) of said air pressure tube (2) and communicate with an inner chamber (22) of said air pressure tube (2).

2. The pressure activated spray head according to claim 1, wherein the upper end (11) of said main body (1) has a spherical shape, and two sliding chutes (111 and 112) are provided in said upper end (11) of said main body (1), said pressure rod (3) disposed in said sliding chute (111) is hinged to said upper end (11) via a pivot pin (31), said spray tube (4) extends through said sliding chute (112) and threadly engages with said pressure head (21) of said air pressure tube (2).

3. The pressure activated spray head according to claim 1, wherein a screw thread is arranged on the inner wall of the connecting sleeve (5).

4. The pressure activated spray head according to claim 2, wherein a screw thread is arranged on the inner wall of the connecting sleeve (5).

5. A pressure activated spray head comprising:
a main body (1), an air pressure tube (2), a pressure rod (3), a spray tube (4) and a connecting sleeve (5), wherein said air pressure tube (2) is inserted into and disposed in said main body (1);
said pressure rod (3) is hinged to said main body and said pressure rod (3) presses against the upper surface of a pressure head (21) of said air pressure tube (2);
a baffle (23) disposed on said air pressure tube (2), said baffle (23) abutting the upper surface of said connecting sleeve (5), and said connecting sleeve (5) embedded in said main body (1);
means for engaging said spray tube (4) with said pressure head (21) of said air pressure tube (2); and
means for connecting said spray tube with an inner chamber (22) of said air pressure tube (2).

6. The pressure activated spray head according to claim 5, wherein the upper end (11) of said main body (1) has a spherical shape, and two sliding chutes (111 and 112) are provided in said upper end (11) of said main body (1), said pressure rod (3) disposed in said sliding chute (111) hinged to said upper end (11) via a pivot pin (31), said spray tube (4) extends through said sliding chute (112) and threadly engages with said pressure head (21) of said air pressure tube (2).

7. The pressure activated spray head according to claim 5, wherein a screw thread is arranged on the inner wall of the connecting sleeve (5).

8. The pressure activated spray head according to claim 6, wherein a screw thread is arranged on the inner wall of the connecting sleeve (5).

9. A pressure activated spray head comprising:
a main body (1), an air pressure tube (2), a pressure rod (3), a spray tube (4) and a connecting sleeve (5), wherein said air pressure tube (2) is inserted into and disposed in said main body (1);
said pressure rod (3) hinged to said main body (1) pressing against the upper surface of a pressure head (21) of said air pressure tube (2);
said spray tube (4) is formed to engage with said pressure head (21) of said air pressure tube (2) and connects with an inner chamber (22) of said air pressure tube (2).

10. The pressure activated spray head according to claim 9, wherein the upper end (11) of said main body (1) includes a first sliding chute (111) and a second sliding chute (112), said pressure rod (3) is disposed in said first sliding chute (111) and hinged to said upper end (11) via a pivot pin (31), said spray tube (4) extends through said second sliding chute (112) and engages with said pressure head (21) of said air pressure tube (2).
